(19) United States
(12) Patent Application Publication
(10) Pub. No.: US 2021/0015449 A1
(43) Pub. Date: Jan. 21, 2021

(54) METHODS AND SYSTEMS FOR PROCESSING AND DISPLAYING FETAL IMAGES FROM ULTRASOUND IMAGING DATA

(71) Applicant: GE Precision Healthcare LLC, Milwaukee, WI (US)

(72) Inventors: Helmut Brandl, Pfaffing (AT); Erwin Fosodeder, Neukirchen an der Vöckla (AT)

(21) Appl. No.: 16/513,582

(22) Filed: Jul. 16, 2019

(51) Int. Cl. A61B 8/08; A61B 5/00; A61B 8/00

(52) U.S. Cl. CPC A61B 8/0866; A61B 5/7267; A61B 8/483; A61B 8/469; A61B 8/5207

(57) ABSTRACT

Various methods and systems are provided for imaging a fetus via an ultrasound imager. In one example, a method may include acquiring imaging data from a probe of an ultrasound imager, generating, from the imaging data, an image slice and a rendering, determining an orientation of the rendering, responsive to determining the orientation not being a standard orientation, adjusting the orientation to the standard orientation, and displaying the image slice unaltered while providing the rendering in the standard orientation.

### FIELD

Embodiments of the subject matter disclosed herein relate to medical imaging, such as ultrasound imaging, and more particularly to processing and displaying fetal images from ultrasound imaging data.

### BACKGROUND

Medical imaging systems are often used to obtain physiological information of a subject. In some examples, the medical imaging system may be an ultrasound system used to obtain and present external physical features of a fetus. In this way, the ultrasound system may be employed to track growth and monitor overall health of the fetus.

Images obtained with the ultrasound system may be presented to a user at a user interface. The user may be a medical professional, and thus the user interface may be configured for use by the medical professional (e.g., displaying vital signs, ultrasound probe controls, and various other user-actuatable functionalities). However, a patient, such as a mother carrying the fetus, may also be presented with the user interface.

### BRIEF DESCRIPTION

In one embodiment, a method may include acquiring imaging data from a probe of an ultrasound imager, generating, from the imaging data, an image slice and a rendering, determining an orientation of the rendering, responsive to determining the orientation not being a standard orientation, adjusting the orientation to the standard orientation, and displaying the image slice unaltered while providing the rendering in the standard orientation.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example ultrasound imaging system according to an exemplary embodiment.

FIG. 2 shows a flow chart of a method for adjusting an orientation of a three-dimensional (3D) rendering of a fetus and displaying the 3D rendering, according to an embodiment.

FIG. 3 shows a flow chart of a method for adjusting the orientation of the 3D rendering in response to a position of a probe of the ultrasound imaging system being altered and displaying the 3D rendering, according to an embodiment.

FIG. 4 shows a flow chart of a method for updating the orientation of the 3D rendering and displaying the 3D rendering, according to an embodiment.

FIG. 5 shows a schematic diagram illustrating an example neural network, according to an embodiment.

FIG. 6 shows a schematic diagram illustrating an example node of the neural network, according to an embodiment.

FIG. 7 shows a schematic diagram of the 3D rendering, according to an embodiment.

FIG. 8A shows a schematic diagram of an example process for maintaining the orientation of the 3D rendering, according to an embodiment.

FIG. 8B shows a schematic diagram of an example process for adjusting the orientation of the 3D rendering, according to an embodiment.

FIG. 9 shows a first example user interface display of a display device of the ultrasound imaging system, according to an embodiment.

FIG. 10 shows a second example user interface display of the display device of the ultrasound imaging system, according to an embodiment.

FIG. 11 shows a schematic diagram of an example process for adjusting a light source relative to the 3D rendering, according to an embodiment.

### DETAILED DESCRIPTION

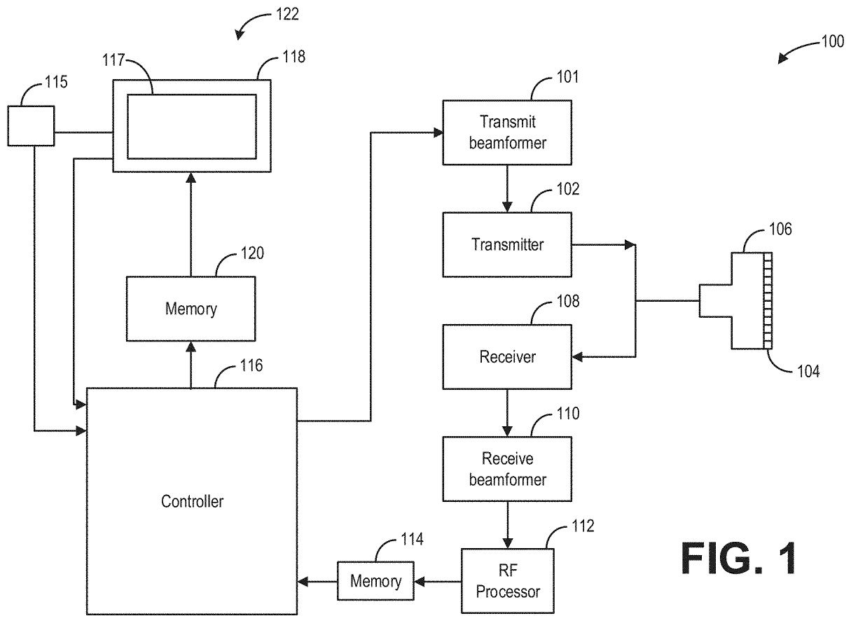

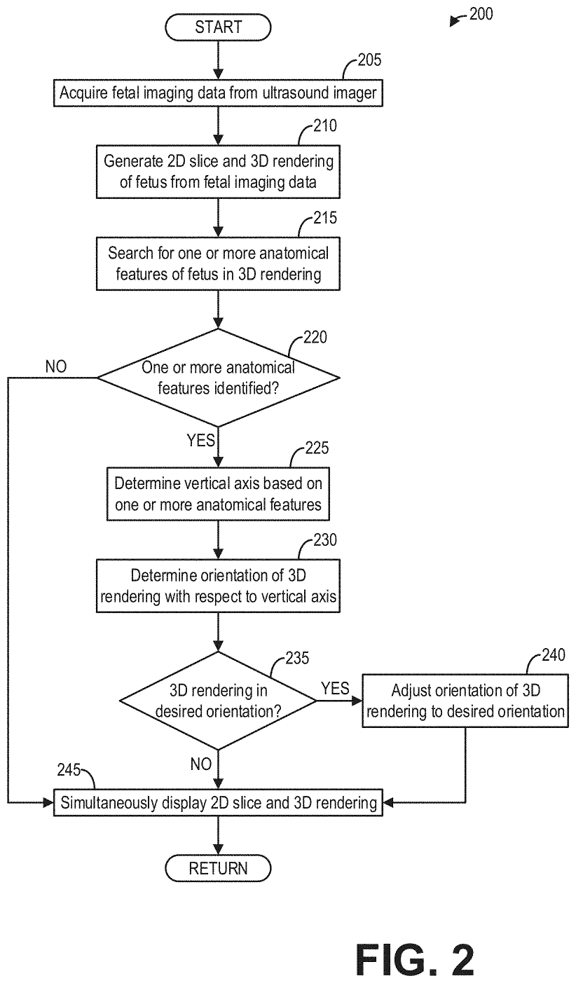

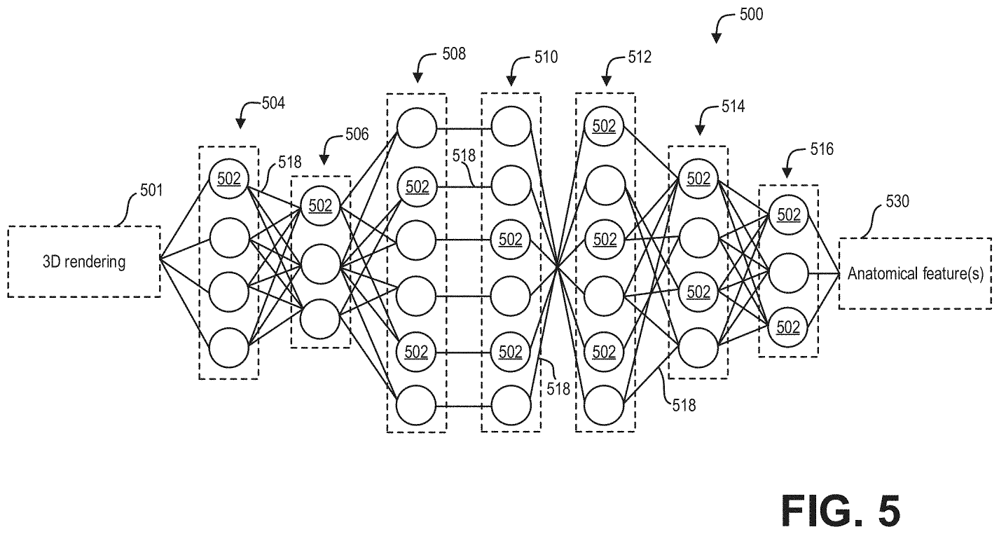

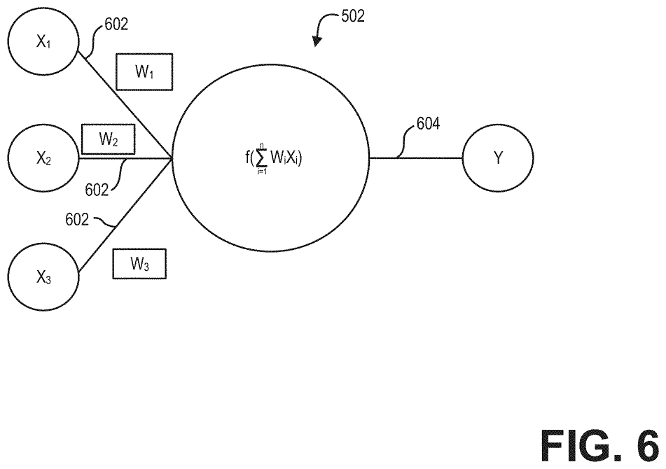

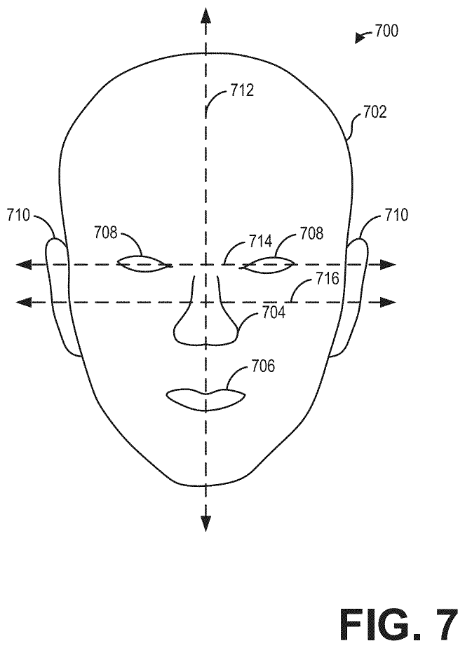

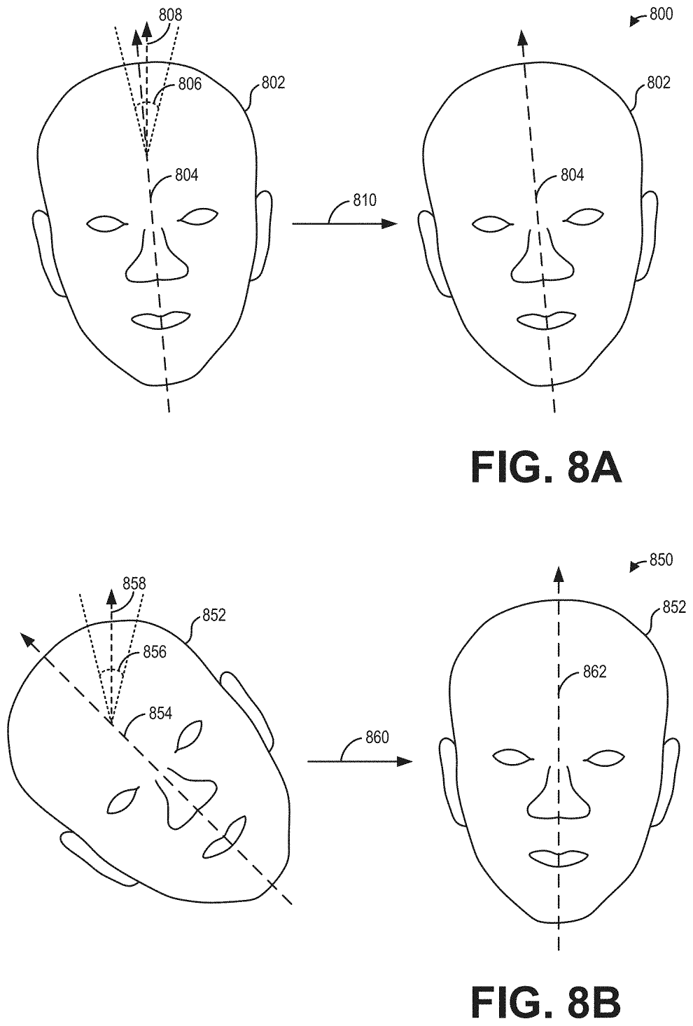

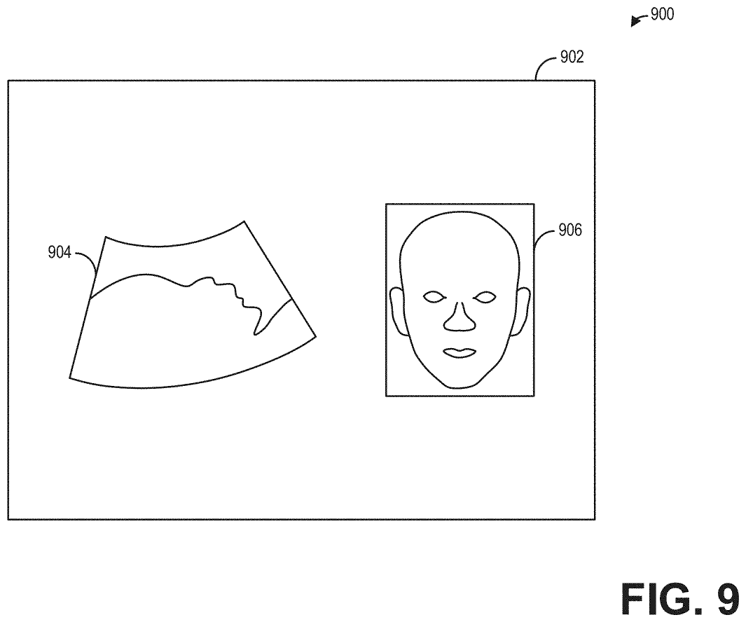

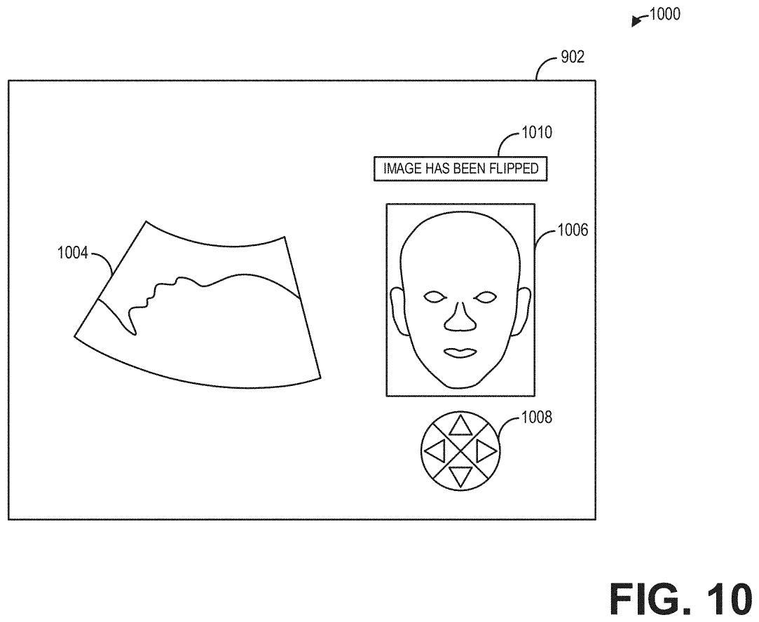

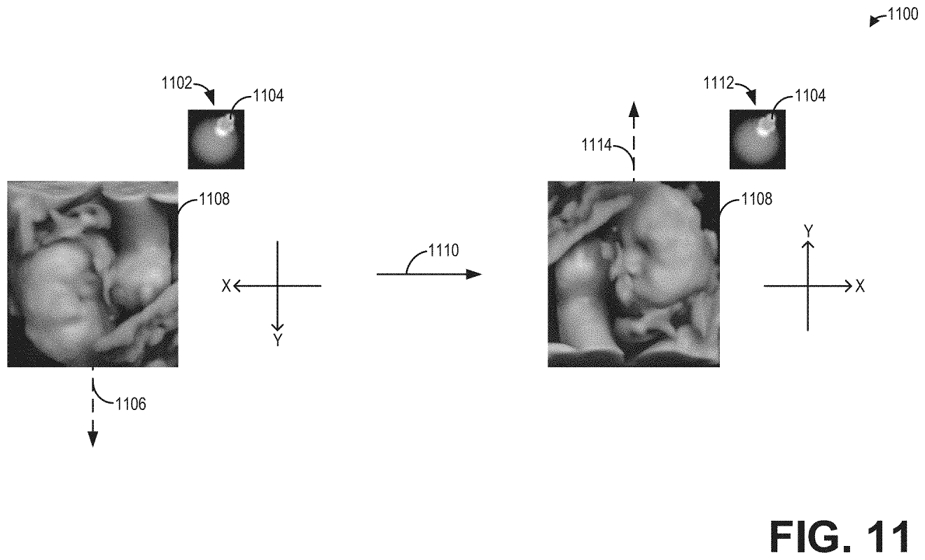

The following description relates to various embodiments of adjusting an orientation of a three-dimensional (3D) rendering of a fetus and displaying the 3D rendering. One example ultrasound imaging system for generating imaging data for the 3D rendering is depicted in FIG. 1. FIGS. 2-4 depict various methods for adjusting the orientation of the 3D rendering and displaying the 3D rendering. An exemplary neural network for recognizing one or more anatomical features depicted by the 3D rendering is depicted by FIGS. 5 and 6. A schematic diagram of the 3D rendering is depicted at FIG. 7. Schematic diagrams of example processes for maintaining and adjusting the orientation of the 3D rendering are depicted at FIGS. 8A and 8B, respectively. Further, a schematic diagram of an example process for adjusting a light source relative to the 3D rendering is depicted at FIG. 11. FIGS. 9 and 10 depict example user interface displays of a display device of the ultrasound imaging system, where a two-dimensional (2D) image slice and the 3D rendering are displayed simultaneously.

FIG. 1 depicts a block diagram of a system 100 according to one embodiment. In the illustrated embodiment, the system 100 is an imaging system and, more specifically, an ultrasound imaging system. However, it is understood that embodiments set forth herein may be implemented using other types of medical imaging modalities (e.g., MR, CT, PET/CT, SPECT etc.). Furthermore, it is understood that other embodiments do not actively acquire medical images. Instead, embodiments may retrieve image or ultrasound data that was previously acquired by an imaging system and analyze the image data as set forth herein. As shown, the system 100 includes multiple components. The components may be coupled to one another to form a single structure, may be separate but located within a common room, or may be remotely located with respect to one another. For example, one or more of the modules described herein may operate in a data server that has a distinct and remote location with respect to other components of the system 100, such as a probe and user interface. Optionally, in the case of ultrasound systems, the system 100 may be a unitary system that is capable of being moved (e.g., portably) from room to room. For example, the system 100 may include wheels or be transported on a cart.

In the illustrated embodiment, the system 100 includes a transmit beamformer 101 and transmitter 102 that drives an array of elements 104, for example, piezoelectric crystals, within a diagnostic ultrasound probe 106 (or transducer) to emit ultrasonic signals (e.g., continuous or pulsed) into a body or volume (not shown) of a subject. The elements 104 and the probe 106 may have a variety of geometries. The ultrasonic signals are back-scattered from structures in a body, for example, facial features of a fetus, to produce echoes that return to the elements 104. The echoes are received by a receiver 108. The received echoes are provided to a receive beamformer 110 that performs beamforming and outputs a radio frequency (RF) signal. The RF signal is then provided to an RF processor 112 that processes the RF signal. Alternatively, the RF processor 112 may include a complex demodulator (not shown) that demodulates the RF signal to form I/Q data pairs representative of the echo signals. The RF or I/Q signal data may then be provided directly to a memory 114 for storage (for example, temporary storage). The system 100 also includes a system controller 116 that may be part of a single processing unit (e.g., processor) or distributed across multiple processing units. The system controller 116 is configured to control operation of the system 100.

For example, the system controller 116 may include an image-processing module that receives image data (e.g., ultrasound signals in the form of RF signal data or I/Q data pairs) and processes image data. For example, the image-processing module may process the ultrasound signals to generate 2D slices or frames of ultrasound information (e.g., ultrasound images) or ultrasound waveforms (e.g., continuous or pulse wave Doppler spectrum or waveforms) for displaying to the operator. Similarly, the image-processing module may process the ultrasound signals to generate 3D renderings of ultrasound information (e.g., ultrasound images) for displaying to the operator. When the system 100 is an ultrasound system, the image-processing module may be configured to perform one or more processing operations according to a plurality of selectable ultrasound modalities on the acquired ultrasound information. By way of example only, the ultrasound modalities may include color-flow, acoustic radiation force imaging (ARFI), B-mode, A-mode, M-mode, spectral Doppler, acoustic streaming, tissue Doppler module, C-scan, and elastography.

Acquired ultrasound information may be processed in real-time during an imaging session (or scanning session) as the echo signals are received. Additionally or alternatively, the ultrasound information may be stored temporarily in the memory 114 during an imaging session and processed in less than real-time in a live or off-line operation. An image memory 120 is included for storing processed slices or waveforms of acquired ultrasound information that are not scheduled to be displayed immediately. The image memory 120 may comprise any known data storage medium, for example, a permanent storage medium, removable storage medium, and the like. Additionally, the image memory 120 may be a non-transitory storage medium.

In operation, an ultrasound system may acquire data, for example, 2D data sets, spectral Doppler data sets, and/or volumetric data sets by various techniques (for example, 3D scanning, real-time 3D imaging, volume scanning, 2D scanning with probes having positioning sensors, freehand scanning using a voxel correlation technique, scanning using 2D or matrix array probes, and the like). Ultrasound spectrum (e.g., waveforms) and/or images may be generated from the acquired data (at the controller 116) and displayed to the operator or user on the display device 118.

The system controller 116 is operably connected to a user interface 122 that enables an operator to control at least some of the operations of the system 100. The user interface 122 may include hardware, firmware, software, or a combination thereof that enables an individual (e.g., an operator) to directly or indirectly control operation of the system 100 and the various components thereof. As shown, the user interface 122 includes a display device 118 having a display area 117. In some embodiments, the user interface 122 may also include one or more user interface input devices 115, such as a physical keyboard, mouse, and/or touchpad. In one embodiment, a touchpad may be configured to the system controller 116 and display area 117, such that when a user moves a finger/glove/stylus across the face of the touchpad, a cursor atop the ultrasound image or Doppler spectrum on the display device 118 moves in a corresponding manner.

In an exemplary embodiment, the display device 118 is a touch-sensitive display (e.g., touchscreen) that can detect a presence of a touch from the operator on the display area 117 and can also identify a location of the touch in the display area 117. The touch may be applied by, for example, at least one of an individual's hand, glove, stylus, or the like. As such, the touch-sensitive display may also be characterized as an input device that is configured to receive inputs from the operator (such as a request to adjust or update an orientation of a displayed image). The display device 118 also communicates information from the controller 116 to the operator by displaying the information to the operator. The display device 118 and/or the user interface 122 may also communicative audibly. The display device 118 is configured to present information to the operator during or after the imaging or data acquiring session. The information presented may include ultrasound images (e.g., one or more 2D slices and 3D renderings), graphical elements, measurement graphics of the displayed images, user-selectable elements, user settings, and other information (e.g., administrative information, personal information of the patient, and the like).

In addition to the image-processing module, the system controller 116 may also include one or more of a graphics module, an initialization module, a tracking module, and an analysis module. The image-processing module, the graphics module, the initialization module, the tracking module, and/or the analysis module may coordinate with one another to present information to the operator during and/or after the imaging session. For example, the image-processing module may be configured to display an acquired image on the display device 118, and the graphics module may be configured to display designated graphics along with the displayed image, such as selectable icons (e.g., image rotation icons) and measurement parameters (e.g., data) relating to the image. The controller may include algorithms and one or more neural networks (e.g., a system of neural networks) stored within a memory of the controller for automatically recognizing one or more anatomical features depicted by a generated ultrasound image, such as a 3D rendering, as described further below with reference to FIGS. 2, 5, and 6. In some examples, the controller may include a deep learning module which includes the one or more deep neural networks and instructions for performing the deep learning and feature recognition discussed herein.

The screen of a display area 117 of the display device 118 is made up of a series of pixels which display the data acquired with the probe 106. The acquired data includes one or more imaging parameters calculated for each pixel, or group of pixels (for example, a group of pixels assigned the same parameter value), of the display, where the one or more calculated image parameters includes one or more of an intensity, velocity (e.g., blood flow velocity), color flow velocity, texture, graininess, contractility, deformation, and rate of deformation value. The series of pixels then make up the displayed image and/or Doppler spectrum generated from the acquired ultrasound data.

The system 100 may be a medical ultrasound system used to acquire imaging data of a scanned object (e.g., a fetus). The acquired image data may be used to generate one or more ultrasound images which may then be displayed via the display device 118 of the user interface 115. The one or more generated ultrasound images may include a 2D image slice and a 3D rendering, for example. For example, the image-processing module discussed above may be programmed to generate and simultaneously display the 2D image slice and the 3D rendering.

In general, during ultrasound imaging of a fetus, the fetus may be in one of a plurality of positions, which may further be in one of a plurality of orientations relative to the ultrasound probe 106. For example, the fetus may be oriented in a non-standard orientation, such as downwards, relative to the ultrasound probe 106 (e.g., where the ultrasound probe is held in a position designated as upside down by a manufacturer). As such, acquired imaging data of the fetus may also result in ultrasound images depicting the fetus in the non-standard orientation. In some examples, the orientation of the acquired imaging data may be adjusted to a standard orientation via manual intervention by a user of the ultrasound probe 106 (e.g., a medical professional). As a first example, a position or orientation of the ultrasound probe 106 may be altered such that acquired imaging data depicts the fetus in the standard orientation relative to the ultrasound probe 106. As a second example, upon display of the ultrasound image at the display device 118, the user may select an icon, which transmits a request to the controller 116 to adjust (e.g., reverse) the orientation of the displayed image. However, manual control of the orientation of the ultrasound images may result in user confusion or patient misinformation in examples where both the user of the ultrasound probe and the patient being examined are presented with the ultrasound images at the display device 118.

According to embodiments disclosed herein, the above-described issues may be at least partly addressed by automatically adjusting an orientation of a generated ultrasound image (e.g., a 3D rendering). Further, in some examples, another generated ultrasound image (e.g., a 2D image slice) may be presented in an acquired orientation (e.g., a non-adjusted orientation), providing a user with further information as to an actual position of a subject (e.g., a fetus) relative to an ultrasound probe. As such, error resulting from mistaken user input, which may further be the result of user confusion, may be minimized, and patient and/or medical professional misinformation may be correspondingly reduced.

Referring now to FIG. 2, a method 200 is depicted for generating a 2D image slice and a 3D rendering from acquired imaging data, e.g., fetal imaging data acquired from an ultrasound imaging system, and then simultaneously displaying the 2D image slice and the 3D rendering, where the 3D rendering may be displayed in a desired, or standard, orientation.

Method 200 is described below with regard to the systems and components depicted in FIG. 1, though it should be appreciated that method 200 may be implemented with other systems and components without departing from the scope of the present disclosure. In some embodiments, method 200 may be implemented as executable instructions in any appropriate combination of the imaging system 100, an edge device (e.g., an external computing device) connected to the imaging system 100, a cloud in communication with the imaging system, and so on. As one example, method 200 may be implemented in non-transitory memory of a computing device, such as the controller (e.g., processor) of the imaging system 100 in FIG. 1.

Method 200 may begin at 205 where fetal imaging data may be acquired from a probe of an ultrasound imager. The ultrasound imager may be one or more components of the imaging system 100 shown in FIG. 1, for example. In such examples, the probe may be the ultrasound probe 106. The probe may be used to image and monitor a fetus. The fetal imaging data may include ultrasound echoes of ultrasound waves transmitted by transducer elements (e.g., elements 104 of FIG. 1) of the probe of the ultrasound imager. In some examples, the imaging data may include volumetric ultrasound data. Further, the volumetric ultrasound data may be based on one or more positional parameters of the ultrasound probe, such as a distance of the ultrasound probe from the fetus and an orientation of the ultrasound probe relative to the fetus. In some examples, the imaging data may further include physiological and/or temporal parameters, sets of multidimensional coordinates, and other information useful for processing the fetal imaging data at an image processing module.

At 210, method 200 may include generating each of a 2D image slice and a 3D rendering depicting the fetus from the fetal imaging data. The 3D rendering may be generated via a ray casting technique, such that the volumetric ultrasound data may be utilized to depict the fetus from a view of the ultrasound probe. For example, the 3D rendering may depict a volume (e.g., from the volumetric ultrasound data) corresponding to an external physical appearance of the fetus. Further, the 2D image slice may correspond to a targeted sagittal slice of the volume (e.g., a profile of a head of the fetus). Each of the 2D image slice and the 3D rendering may be generated with a default, or acquired, orientation resulting from the orientation of the ultrasound probe relative to the fetus.

The 3D rendering may be shaded in order to present a user with a better perception of depth. This may be performed in several different ways according to various embodiments. For example, a plurality of surfaces may be defined based on the volumetric ultrasound data and/or voxel data may be shaded via ray casting. According to an embodiment, a gradient may be calculated at each pixel. The controller 116 (shown in FIG. 1) may compute an amount of light at positions corresponding to each pixel and apply one or more shading methods based on the gradients and specific light directions. A view direction may correspond with a standard view direction, such as angled from above the 3D rendering. The controller 116 may also use multiple light sources as inputs when generating the 3D rendering.

In an example, when ray casting, the controller 116 may calculate how much light is reflected, scattered, or transmitted from each voxel in a particular view direction along each ray. This may involve summing contributions from multiple light sources (e.g., point light sources). The controller 116 may calculate contributions from all voxels in the volume. The controller 116 may then composite values from all voxels, or interpolated values from neighboring voxels, in order to compute a final value of a displayed pixel on the 3D rendering. While the aforementioned example describes an embodiment where voxel values are integrated along rays, 3D renderings may also be calculated according to other techniques such as using a highest value along each ray, using an average value along each ray, or using any other volume-rendering technique.

At 215, method 200 may include searching for one or more anatomical features of the fetus depicted in the 3D rendering. The one or more anatomical features may include external physical features of the fetus, such as limbs. In some examples, the one or more anatomical features may include one or more facial features, such as a nose, a mouth, one or both eyes, one or both ears, etc. In some examples, facial recognition algorithms may be employed to search for, and subsequently automatically identify, the one or more facial features. Such facial recognition algorithms may include a deep neural network, or system of deep neural networks, such as the example neural network described with reference to FIGS. 5 and 6. In some examples, the deep neural network, or system of deep neural networks, may be trained with a training set of additional 3D renderings prior to identifying the one or more facial features from the generated 3D rendering.

At 220, method 200 may include automatically determining whether the one or more anatomical features have been identified. If the one or more anatomical features have not been identified, e.g., if the facial recognition algorithm has not recognized one or more facial features, method 200 may proceed to 245 to simultaneously display the 2D image slice and the 3D rendering. In such examples, each of the 2D image slice and the 3D rendering may be displayed in the acquired orientations, as described hereinabove. Method 200 may then end.

If the one or more anatomical features have been identified, e.g., if the facial recognition algorithm has returned coordinates corresponding to one or more facial features, method 200 may proceed to 225 to determine a vertical axis based on the one or more anatomical features. In some examples, “vertical axis” may refer to a bidirectional axis parallel to a line bifurcating a face of the fetus along the nose, mouth, chin, forehead, etc. In some examples, the vertical axis may be determined by first determining a transverse axis based on the one or more anatomical features. In some examples, “transverse axis” may refer to a bidirectional axis parallel to a line bifurcating each of the eyes or each of the ears. As such, the vertical axis may be generated as an axis perpendicular to the transverse axis which bifurcates a further facial feature (e.g., the nose or the mouth). Further examples are described hereinbelow with reference to FIG. 7.

At 230, method 200 may include determining an orientation of the 3D rendering with respect to the vertical axis. For example, the orientation of the 3D rendering may be represented by a first vector parallel to the vertical axis, and directed in a standard direction, such as directed from the mouth to the nose to the forehead. In this way, the orientation of the 3D rendering may be automatically determined based on the vertical axis and the one or more identified anatomical features. A second vector may further be defined directed in a standard direction relative to the ultrasound probe. For example, the standard direction of the second vector may be a default, upwards direction relative to the ultrasound probe (e.g., wherein the ultrasound probe may be assumed to be held in a position designated as upright by a manufacturer).

At 235, method 200 may include determining whether the 3D rendering is in a desired orientation. For example, the desired orientation may include a standard orientation of the 3D rendering (e.g., where the fetus is depicted in an upright position relative to a display device, such as where a head of the fetus is depicted above a torso of the fetus, or where the nose of the fetus is depicted above the mouth of the fetus). In examples wherein the second vector is defined as the desired, or standard, orientation, determining whether the 3D rendering is in the desired orientation may include determining whether the determined orientation (e.g., the first vector) of the 3D rendering is within a threshold angle (e.g., less than 30°, 20°, or 10°) of the second vector. Exemplary embodiments of a process of determining whether the 3D rendering is in the desired orientation are described hereinbelow with reference to FIGS. 8A and 8B.

If the 3D rendering is in the desired orientation (e.g., if the determined angle between the first vector and the second vector is within the threshold angle of the second vector), method 200 may proceed to 245 to simultaneously display the 2D image slice and the 3D rendering, where the 3D rendering may be displayed and maintained in the determined orientation. In such examples, the determined orientation may be considered the desired, or standard, orientation. In some examples, the 2D image slice may be displayed in an acquired orientation. Method 200 may then end.

If the 3D rendering is not in the desired orientation (e.g., if the determined angle between the first vector and the second vector is outside of the threshold angle of the second vector), method 200 may proceed to 240 to automatically adjust the determined orientation of the 3D rendering to the desired, or standard, orientation. In some examples, automatically adjusting the determined orientation may include rotating the 3D rendering about a rotation axis mutually perpendicular to the vertical axis and the transverse axis until the second vector is both parallel to the first vector and is oriented in a same direction as the first vector. That is, in such examples, rotation of the 3D rendering may not be performed about the vertical axis used to determine the orientation of the 3D rendering or the transverse axis used to determine the vertical axis. In additional or alternative examples, automatically adjusting the determined orientation may include automatically reversing the determined orientation of the 3D rendering (e.g., rotating the 3D rendering 180° about the rotation axis). In other examples, automatically adjusting the determined orientation of the 3D rendering may instead include rotating the volume represented by the volumetric data in a similar manner and then generating a new 3D rendering in the desired orientation based on the rotated volume.

In some examples, automatically adjusting the determined orientation of the 3D rendering may further include automatically adjusting or maintaining positions of one or more light sources (e.g., point light sources) of the 3D rendering and thus re-shading the rendered image as compared to a different orientation. For example, the one or more light sources may be in initial positions relative to the 3D rendering in the determined orientation. The initial positions may be default positions lighting the 3D rendering as though from above, for example (e.g., simulating sunlight or an overhead light in a room). Upon automatic adjustment of the determined orientation of the 3D rendering to the desired orientation, the one or more light sources may remain fixed in the initial positions such that the 3D rendering may be lit in a desirable manner. In other examples, the one or more light sources may be adjusted from the initial positions to provide desirable lighting of the 3D rendering in the adjusted orientation. An exemplary embodiment of a process of adjusting an example light source relative to the 3D rendering is described hereinbelow with reference to FIG. 11. It should be appreciated that the shading control as described herein may provide significant technical advantages. Specifically, if the image is rendered in the uncorrected orientation, in combination with shading that expects a proper orientation, it can be especially difficult for a user to recognize that the image is in the improper or undesired orientation (as illustrated in FIG. 11). This is because the shading is coming from a direction that makes recognizing the 3D rendering even more difficult for a user as the shading is unfamiliar as users typically recognize facial features with shading from above, not below, for example. Thus, by identifying the proper orientation from the 3D data (which is unshaded), the system is better able to identify the proper orientation and not only display the image in a more recognizable orientation, but also with shading from the more recognizable (to the user) direction.

In this way, the acquired orientation of the 3D rendering may be automatically adjusted to the desired orientation according to one or more identified anatomical features. Method 200 may proceed to 245 to simultaneously display the 2D image slice and the 3D rendering, where the 3D rendering may be displayed in the adjusted orientation. In such examples, the adjusted orientation may be considered the desired, or standard orientation.

In some examples, the 2D image slice may be displayed in the acquired orientation thereof. In some examples, the acquired orientation of the 2D image slice may include depicting the sagittal slice of the head of the fetus in a leftwards or rightwards orientation, which may correspond to an upwards and downwards orientation of the head of the fetus in the 3D rendering, respectively. As such, a user of the ultrasound imaging system may infer whether the orientation of the 3D rendering has been automatically adjusted. For example, if the 2D image slice is displayed in the rightwards orientation and the 3D rendering is displayed in the upwards orientation, then the user of the ultrasound imaging system may infer that the 3D rendering has been automatically adjusted from the downwards orientation. In some examples, a notification or an alert may further be displayed when the orientation of the 3D rendering has been automatically adjusted based on the one or more identified anatomical features. In additional or alternative examples, an initial color channel of the displayed 3D rendering may be altered when the orientation of the 3D rendering has been automatically adjusted. For example, the displayed 3D rendering may be initially displayed in the initial color channel, such as tan monochrome, by default, and may be displayed in an altered color channel, such as gray monochrome, when the orientation of the 3D rendering has been automatically adjusted. Two examples of such displays are provided hereinbelow with reference to FIGS. 9 and 10. In this way, the user (e.g., a medical professional) of the ultrasound imaging system may be provided with an equivalent amount of physiological and location-based information of the fetus even if displayed images of the fetus have been adjusted to a standard orientation. Method 200 may then end.

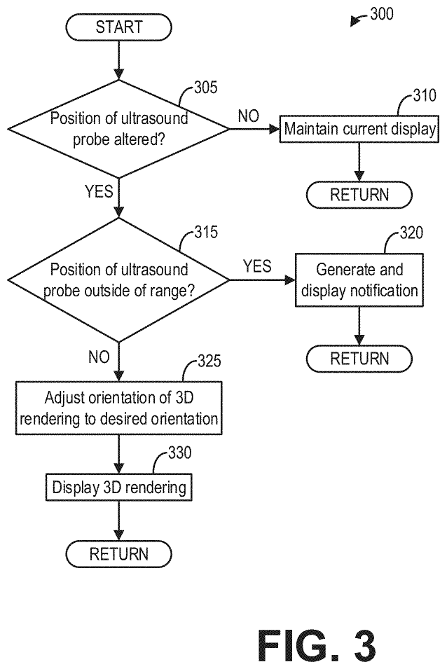

Referring now to FIG. 3, a method 300 is depicted for adjusting the orientation of the 3D rendering in response to a position of the ultrasound imaging probe being altered, and then displaying the 3D rendering in the adjusted orientation. In some examples, adjusting the orientation of the 3D rendering may include adjusting the orientation of the 3D rendering to a desired, or standard, orientation. In some examples, method 300 may follow method 200. As such, in some examples, the 3D rendering may initially be displayed in the desired, or standard, orientation.

Method 300 is described below with regard to the systems and components depicted in FIG. 1, though it should be appreciated that method 300 may be implemented with other systems and components without departing from the scope of the present disclosure. In some embodiments, method 300 may be implemented as executable instructions in any appropriate combination of the imaging system 100, an edge device (e.g., an external computing device) connected to the imaging system 100, a cloud in communication with the imaging system, and so on. As one example, method 300 may be implemented in non-transitory memory of a computing device, such as the controller (e.g., processor) of the imaging system 100 in FIG. 1.

Method 300 may begin at 305, where method 300 may determine whether a position of the ultrasound probe has been altered (e.g., following an initial generation and display of one or more ultrasound images). For example, the position of the ultrasound probe may be manually altered by a user of the ultrasound probe (e.g., a medical professional). If the position of the ultrasound probe has not been altered, method 300 may proceed to 310 to maintain a current display. For example, the current display may include the 2D image slice and the 3D rendering as generated and displayed according to method 200, as described above with reference to FIG. 2. Method 300 may then end.

If the position of the ultrasound probe has been altered, method 300 may proceed to 315 to determine whether the altered position of the ultrasound probe is outside of a detection range of a fetus. For example, one or more anatomical features of the fetus may have been previously identified and are subsequently determined to no longer be present in imaging data received from the ultrasound probe in the altered position (e.g., via the neural network of FIGS. 5 and 6). As such, if the altered position of the ultrasound probe is outside of the detection range, method 300 may proceed to 320 to generate and display a notification or an alert. The notification may indicate to the user of the ultrasound imaging system that the ultrasound probe is outside of the detection range of the fetus. In some examples, the notification may include a prompt indicating that the altered position of the ultrasound probe should be manually adjusted to back within the detection range of the fetus. Further, in some examples, the 2D image slice and the 3D rendering may continue to be displayed and may change in appearance in response to newly received fetal imaging data (e.g., in response to the ultrasound probe being moved). However, in such examples, the orientation of the 3D rendering may not be automatically adjusted. Method 300 may then end.

If the altered position of the ultrasound probe is within the detection range, method 300 may proceed to 325 to automatically adjust the orientation of the 3D rendering to the desired, or standard, orientation. In some examples, an analogous procedure to that described at 215 to 245 of method 200 as described in FIG. 2 may be employed to automatically adjust the orientation of the 3D rendering to the desired orientation. In examples wherein the orientation of the 3D rendering has previously been adjusted to the desired orientation (e.g., via method 200 of FIG. 2), method 300 may be considered to re-adjust, or correct, the orientation following the position of the ultrasound probe being altered. In examples wherein processing power of the ultrasound imaging system is lower or rendering demands are higher, a delay may occur between receipt of the fetal imaging data (e.g., from movement of the ultrasound probe) and generation of the 3D rendering. In such examples, the orientation and display of the 3D rendering may be adjusted only following the delay. In other examples, the ultrasound imaging system may enable automatic adjustment of the orientation of the 3D rendering in real time upon movement of the ultrasound probe. In this way, the orientation of the 3D rendering may automatically be adjusted to the desired, or standard, orientation when the position of the ultrasound probe is altered.

At 330, method 300 may include displaying the 3D rendering in the desired, or standard, orientation. Method 300 may then end.

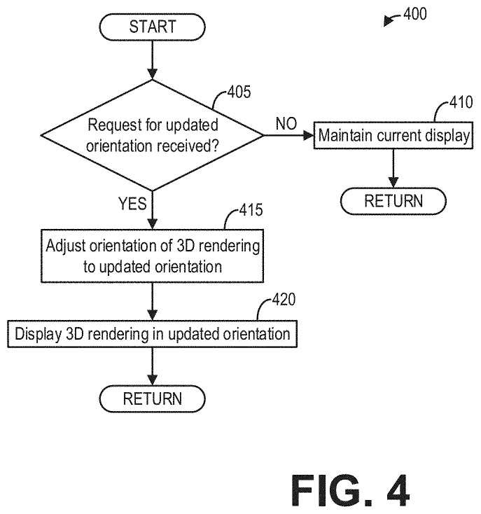

Referring now to FIG. 4, a method 400 is depicted for updating the orientation of the 3D rendering and then displaying the 3D rendering in the updated orientation. In some examples, updating the orientation of the 3D rendering may be in response to a user request for the updated orientation. In some examples, method 400 may follow method 200. As such, in some examples, the 3D rendering may initially be displayed in the desired, or standard, orientation.

Method 400 is described below with regard to the systems and components depicted in FIG. 1, though it should be appreciated that method 400 may be implemented with other systems and components without departing from the scope of the present disclosure. In some embodiments, method 400 may be implemented as executable instructions in any appropriate combination of the imaging system 100, an edge device (e.g., an external computing device) connected to the imaging system 100, a cloud in communication with the imaging system, and so on. As one example, method 400 may be implemented in non-transitory memory of a computing device, such as the controller (e.g., processor) of the imaging system 100 in FIG. 1.

Method 400 may begin at 405, where method 400 may include determining whether a request (e.g., a user request) for the updated orientation of the 3D rendering has been received. In some examples, the updated orientation may be requested (e.g., by a user) via an icon at the display device (e.g., the display device 118 of FIG. 1). For example, the icon may provide options for rotating the orientation of the 3D rendering by 90°, 180°, and/or 270°. In additional or alternative examples, the request may be a user-inputted value for an angle of rotation. If no request for the updated orientation has been received, method 400 may proceed to 410 to maintain a current display. For example, the current display may include the 2D image slice and the 3D rendering as generated and displayed according to method 200, as described above with reference to FIG. 2. Method 400 may then end.

If the request for the updated orientation has been received, method 400 may proceed to 420 to automatically adjust the orientation of the 3D rendering to the updated orientation. In some examples, an analogous procedure to that described at 215 to 245 of method 200 as described in FIG. 2 may be employed to automatically adjust the orientation of the 3D rendering to the updated orientation. In some examples, the updated orientation may not be the standard orientation. In such examples, the request for the updated orientation may override automatic adjustment of the orientation of the 3D rendering to the standard orientation. In some examples, the orientation of the 3D rendering may remain at the updated orientation for a set amount of time. In additional or alternative examples, the orientation of the 3D rendering may remain at the updated orientation until a user has finished using the ultrasound imaging system (e.g., until the ultrasound imaging system is shut off). In this way, the orientation of the 3D rendering may automatically be adjusted to the updated orientation and thereby permit manual override of the ultrasound imaging system via a user request.

At 420, method 400 may include displaying the 3D rendering in the updated orientation. Method 400 may then end.

Referring now to FIGS. 5 and 6, an exemplary neural network for identifying and classifying one or more anatomical features from a 3D rendering input of a subject, such as a fetus, is depicted. In some examples, the neural network may be trained with a training set of additional 3D renderings depicting other fetuses prior to identifying the one or more anatomical features from the 3D rendering input. In examples wherein the one or more anatomical features include one or more facial features, the neural network may be considered a facial recognition algorithm.

FIG. 5 depicts a schematic diagram of a neural network 500 having one or more nodes/neurons 502 which, in some embodiments, may be disposed into one or more layers 504, 506, 508, 510, 512, 514, and 516. Neural network 500 may be a deep neural network. As used herein with respect to neurons, the term “layer” refers to a collection of simulated neurons that have inputs and/or outputs connected in similar fashion to other collections of simulated neurons. Accordingly, as show in FIG. 5, neurons 502 may be connected to each other via one or more connections 518 such that data may propagate from an input layer 504, through one or more intermediate layers 506, 508, 510, 512, and 514, to an output layer 516.

FIG. 6 shows input and output connections for a neuron in accordance with an exemplary embodiment. As shown in FIG. 6, connections (e.g., 518) of an individual neuron 502 may include one or more input connections 602 and one or more output connections 604. Each input connection 602 of neuron 502 may be an output connection of a preceding neuron, and each output connection 604 of neuron 502 may be an input connection of one or more subsequent neurons. While FIG. 6 depicts neuron 502 as having a single output connection 604, it should be understood that neurons may have multiple output connections that send/transmit/pass the same value. In some embodiments, neurons 502 may be data constructs (e.g., structures, instantiated class objects, matrices, etc.) and input connections 602 may be received by neuron 502 as weighted numerical values (e.g., floating point or integer values). For example, as further shown in FIG. 6, input connections X₁, X₂, and X₃ may be weighted by weights W₁, W₂, and W₃, respectively, summed, and sent/transmitted/passed as output connection Y. As will be appreciated, the processing of an individual neuron 502 may be represented generally by the equation:

$$Y = f ( {\sum }_{i = 1}^{n} {W}_{i} {X}_{i} )$$

where n is the total number of input connections 602 to neuron 502. In one embodiment, the value of Y may be based at least in part on whether the summation of WᵢXᵢ exceeds a threshold. For example, Y may have a value of zero (0) if the summation of the weighted inputs fails to exceed a desired threshold.

As will be further understood from FIGS. 5 and 6, input connections 602 of neurons 502 in input layer 504 may be mapped to an input 501, while output connections 604 of neurons 502 in output layer 516 may be mapped to an output 530. As used herein, “mapping” a given input connection 602 to input 501 refers to the manner by which input 501 affects/dictates the value said input connection 602. Similarly, as also used herein, “mapping” a given output connection 604 to output 530 refers to the manner by which the value of said output connection 604 affects/dictates output 530.

Accordingly, in some embodiments, the acquired/obtained input 501 is passed/fed to input layer 504 of neural network 500 and propagated through layers 504, 506, 508, 510, 512, 514, and 516 such that mapped output connections 604 of output layer 516 generate/correspond to output 530. As shown, input 501 may include a 3D rendering of a subject, such as a fetus, generated from ultrasound imaging data. The 3D rendering may depict a view of the fetus showing one or more anatomical features (such as one or more facial features, e.g., a nose, a mouth, eyes, ears, etc.) identifiable by the neural network 500. Further, output 530 may include locations and classifications of one or more identified anatomical features depicted in the 3D rendering. For example, the neural network 500 may identify an anatomical feature depicted by the rendering, generate coordinates indicating a location (e.g., a center, a perimeter) of the anatomical feature, and classify the anatomical feature (e.g., as a nose) based on identified visual characteristics. In examples wherein the neural network 500 is a facial recognition algorithm, output 530 may specifically include one or more facial features.

Neural network 500 may be trained using a plurality of training datasets. Each training dataset may include additional 3D renderings depicting one or more anatomical features of further fetuses. Thus, the neural network 500 may learn relative positioning and shapes of the one or more anatomical features depicted in the 3D renderings. In this way, neural network 500 may utilize the plurality of training datasets to map generated 3D renderings (e.g., inputs) to one or more anatomical features (e.g., outputs). The machine learning, or deep learning, therein (due to, for example, identifiable trends in placement, size, etc. of anatomical features) may cause weights (e.g., W₁, W₂, and/or W₃) to change, input/output connections to change, or other adjustments to neural network 500. Further, as additional training datasets are employed, the machine learning may continue to adjust various parameters of the neural network 500 in response. As such, a sensitivity of the neural network 500 may be periodically increased, resulting in a greater accuracy of anatomical feature identification.

Referring now to FIG. 7, a schematic diagram depicts an example 3D rendering 700 of one or more anatomical features of a subject. In the example shown, the 3D rendering 700 specifically depicts at least a face 702 of the fetus, where the face 702 has one or more facial features. The one or more facial features may be automatically identified by a system, such as the system 100 as described with reference to FIG. 1, implementing a neural network or other artificial intelligence routine, such as the neural network 500 as described with reference to FIGS. 5 and 6. The one or more identified facial features may then be used to automatically determine one or more axes of the 3D rendering 700, from which an orientation of the 3D rendering 700 may be further determined.

For example, the 3D rendering 700 may depict a nose 704 and a mouth 706. Upon automatic identification of the nose 704 and the mouth 706, a vertical axis 712 may be generated. The vertical axis 712 may be defined as bifurcating each of the nose 704 and the mouth 706. Further, the relative positions of the nose 704 and the mouth 706 may provide further information as to the orientation of the 3D rendering 700 (e.g., in which direction the face 702 of the fetus is oriented).

As another example, wherein only one of the nose 704 and the mouth 706 are identified, eyes 708 may be further identified. Upon identification of the eyes 708, a transverse axis 714 may be generated. The transverse axis 714 may be defined as bifurcating each of the eyes 708. After the transverse axis 714 has been identified, the vertical axis 712 may be defined as bifurcating the one of the nose 704 and the mouth 706 identified, and as being perpendicular to the transverse axis 714. Further, the relative positions of the eyes 708 and the one of the nose 704 and the mouth 706 may provide further information as to the orientation of the 3D rendering 700.

As yet another example, wherein only one of the nose 704 and the mouth 706 are identified, ears 710 may be further identified. Upon identification of the ears 710, a transverse axis 716 may be generated. The transverse axis 716 may be defined as bifurcating each of the ears 710. After the transverse axis 716 has been identified, the vertical axis 712 may be defined as bifurcating the one of the nose 704 and the mouth 706 identified, and as being perpendicular to the transverse axis 716. Further, the relative positions of the ears 710 and the one of the nose 704 and the mouth 706 may provide further information as to the orientation of the 3D rendering 700.

It will be understood by those skilled in the art that there are numerous methods of geometrically determining two points with which to define the vertical axis 712, and that the examples presented herein are not to be considered as limiting embodiments.

Referring now to FIG. 8A, a schematic diagram of an example process 800 for automatically maintaining an orientation 804 of an example 3D rendering 802 is depicted. In some examples, a method, such as method 200 as described with reference to FIG. 2, may be implemented on a system, such as system 100 as described with reference to FIG. 1, to automatically determine the orientation 804 of the 3D rendering 802. Further, a threshold angle 806 may be set with reference to a vector 808, where the vector 808 aligns with a standard orientation of the 3D rendering 802. For example, the threshold angle 806 may be 20°, indicating that a given determined orientation (e.g., 804) within 20° of the vector 808 may be determined as being, or approximately being, the standard orientation. As such, upon determination of the orientation 804 being within the threshold angle 806, the orientation 804 may be maintained 810 such that the orientation 804 is not altered. As shown in FIG. 8A, the orientation 804 may not precisely align with the standard orientation (e.g., the orientation 804 may not be parallel with the vector 808), but may still be considered close enough to the standard orientation that no adjustment action is taken.

Referring now to FIG. 8B, a schematic diagram of an example process 850 for automatically adjusting an orientation 854 of an example 3D rendering 852 is depicted. In some examples, a method, such as method 200 as described with reference to FIG. 2, may be implemented on a system, such as system 100 as described with reference to FIG. 1, to automatically determine the orientation 854 of the 3D rendering 852. Further, a threshold angle 856 may be set with reference to a vector 858, where the vector 858 aligns with a standard orientation of the 3D rendering 852. For example, the threshold angle 856 may be 20°, indicating that a given determined orientation (e.g., 854) within 20° of the vector 858 may be determined as being, or approximately being, the standard orientation. As such, upon determination of the orientation 854 being outside of the threshold angle 856, the orientation 854 may be adjusted 860 to an adjusted orientation 862 such that the orientation 854 is altered to align with the standard orientation (e.g., the orientation 854 is adjusted to be parallel with the vector 858).

Referring now to FIG. 11, a schematic diagram of an example process 1100 for automatically adjusting each of a position 1102 of an example light source 1104 and an orientation 1106 of an example 3D rendering 1108 is depicted. In the depicted example, the light source 1104 may be a point light source. In some examples, a method, such as method 200 as described with reference to FIG. 2, may be implemented on a system, such as system 100 as described with reference to FIG. 1, to automatically determine each of the orientation 1106 of the 3D rendering 1108 and the position 1102 of the light source 1104. For reference, positive X and Y directions further contextualize the position 1102 of the light source 1104 and the orientation 1106 of the 3D rendering 1108.

As shown, the position 1102 of the light source 1104 may be such that a fetus depicted in the 3D rendering 1108 is lit from an angle below a face of the fetus. Such lighting may be contrary to user expectations, as faces may often be lit from above (e.g., via sunlight or an overhead light in a room). Thus, one or more facial features of the depicted fetus may be more difficult for a user of the system (e.g., 100) to recognize, due not only to the orientation 1106 of the 3D rendering 1108 being in a non-standard direction, for example, but also due to the one or more facial features being non-intuitively shadowed in the 3D rendering 1108 because of the position 1102 of the light source 1104.

Each of the position 1102 of the light source 1104 and the orientation 1106 of the 3D rendering 1108 may be adjusted 1110 to an adjusted position 1112 of the light source 1104 and an adjusted orientation 1114 of the 3D rendering 1108. Though the adjusted position 1112 may appear the same as the position 1102, the positive X and Y directions have also been adjusted in the schematic diagram 1100 so as to clearly indicate that the adjusted position 1112 is indeed altered relative to the 3D rendering 1108. As shown in the rendering 1108, shadowing of the one or more facial features of the depicted fetus has been altered due to the adjusted position 1112 of the light source 1102 such that the one or more facial features may be more recognizable to the user of the system (e.g., 100). In this way, a lighting and an orientation of a 3D rendering depicting one or more facial features of a fetus may be automatically adjusted when the one or more facial features may be difficult to recognize by a user of an ultrasound imaging system, precluding ease of manual adjustment of the lighting and/or the orientation of the 3D rendering.

Referring now to FIG. 9, a first example user interface display 900 of a display device 902 is depicted. In one example, the display device 902 may be the display device 118 shown in FIG. 1. The first example user interface display 900 may include a simultaneous display of an example 2D image slice 904 and an example 3D rendering 906, where an orientation of the 3D rendering 906 is a standard orientation. An orientation of the 2D image slice 904 may be an acquired, or initial, orientation, as set following generation of the 2D image slice 904 from imaging data. As shown, the orientation of the 2D image slice 904 depicts a head of a fetus on the left, which, in some examples, may indicate to a user that the 3D rendering 906 is also displayed in an acquired orientation. In such examples, the acquired orientation of the 3D rendering 906 may be an upwards orientation relative to the display device 902. As such, the first example user interface display 900 may be displayed at the display device 902 following the orientation of the 3D rendering 906 being maintained at the acquired orientation, such as in the process described above with reference to FIG. 8A. In some examples, each of the 2D image slice 904 and the 3D rendering 906 may be selectable via a user of the ultrasound imaging system (e.g., via touch, a mouse, a keyboard, etc.) for further user manipulation.

Referring now to FIG. 10, a second example user interface display 1000 of the display device 902 is depicted. The second example user interface display 1000 may include a simultaneous display of an example 2D image slice 1004 and an example 3D rendering 1006, where an orientation of the 3D rendering 1006 is a standard orientation. An orientation of the 2D image slice 1004 may be an acquired, or initial, orientation, as set following generation of the 2D image slice 1004 from imaging data. As shown, the orientation of the 2D image slice 1004 depicts a head of a fetus on the right, which, in some examples, may indicate to a user that the 3D rendering 1006 has been automatically adjusted to the standard orientation. In such examples, the standard orientation of the 3D rendering 1006 may be an upwards orientation relative to the display device 902. As such, the second example user interface display 1000 may be displayed at the display device 902 following the orientation of the 3D rendering 1006 being adjusted to the standard orientation, such as in the process described above with reference to FIG. 8B.

In some examples, the orientation of the 3D rendering 1006 may be updated upon receiving a user request at a user interface of an ultrasound imaging system, such as the user interface 115 of the ultrasound imaging system 100 shown in FIG. 1. The user request may be input, for example, via an image rotation icon 1008. As shown, the image rotation icon 1008 may provide options for rotating the orientation of the 3D rendering by 90°, 180°, and 270°. In additional or alternative examples, the user request may be a user-inputted value for an angle of rotation. In some examples, each of the 2D image slice 1004, the 3D rendering 1006, and the image rotation icon 1008 may be selectable via a user of the ultrasound imaging system (e.g., via touch, a mouse, a keyboard, etc.) for further user manipulation. Further, in some examples a notification 1010 may be displayed when the orientation of the 3D rendering 1006 has been automatically adjusted. In this way, the user may be provided with multiple sources of information for inferring and controlling the orientation of the 3D rendering relative to an ultrasound probe (e.g., 106).

In this way, an orientation a three-dimensional (3D) rendering of a fetus generated from ultrasound imaging data may be automatically adjusted based on identification of one or more anatomical features of the fetus. In one example, the one or more anatomical features may be one or more facial features used to determine a vertical axis of the fetus, from which the orientation of the 3D rendering may be determined. A technical effect of using one or more facial features in adjusting the orientation of the 3D rendering is that a facial recognition algorithm may be employed to identify the one or more facial features, which may provide multiple points of reference with which to define the vertical axis aligned with the orientation. Further, after the orientation is adjusted, the 3D rendering may be provided with an unaltered two-dimensional (2D) image slice of the fetus at a user interface display. A technical effect of simultaneously displaying the 2D image slice and the 3D rendering in this way is that a user may be provided with sufficient information to infer an actual orientation of a probe of an ultrasound imager providing the ultrasound imaging data even following automatic adjustment of the orientation of the 3D rendering.

In one embodiment, a method comprises acquiring imaging data from a probe of an ultrasound imager, generating, from the imaging data, an image slice and a rendering, determining an orientation of the rendering, responsive to determining the orientation not being a standard orientation, adjusting the orientation to the standard orientation, and displaying the image slice unaltered while providing the rendering in the standard orientation. In a first example of the method, the imaging data includes fetal imaging data, and each of the image slice and the rendering depict one or more anatomical features of a fetus. In a second example of the method, optionally including the first example, the one or more anatomical features comprise one or more facial features, and the standard orientation is an upwards orientation relative to a vertical axis determined from the one or more facial features. In a third example of the method, optionally including one or more of the first and second examples, determining the orientation of the rendering includes identifying the one or more anatomical features, and determining the orientation based on the one or more identified anatomical features. In a fourth example of the method, optionally including one or more of the first through third examples, identifying the one or more anatomical features includes using a system of deep neural networks to identify the one or more anatomical features from the rendering. In a fifth example of the method, optionally including one or more of the first through fourth examples, prior to identifying the one or more anatomical features from the rendering, the system of deep neural networks is trained with a training set of additional renderings depicting one or more anatomical features of further fetuses. In a sixth example of the method, optionally including one or more of the first through fifth examples, the method further comprises, responsive to a position of the probe of the ultrasound imager being altered, automatically adjusting the orientation to the standard orientation in real time. In a seventh example of the method, optionally including one or more of the first through sixth examples, the method further comprises, responsive to a position of the probe of the ultrasound imager being altered, responsive to the altered position being outside a detection range of the fetus, generating and displaying a notification, and responsive to the altered position being inside the detection range of the fetus, adjusting the orientation to the standard orientation. In a eighth example of the method, optionally including one or more of the first through seventh examples, the method further comprises receiving a user request for an updated orientation, adjusting the orientation to the updated orientation, and displaying the rendering in the updated orientation.

In another embodiment, a system comprises an ultrasound probe, a user interface configured to receive input from a user of the system, a display device, and a processor configured with instructions in non-transitory memory that when executed cause the processor to acquire fetal imaging data from the ultrasound probe, generate, from the fetal imaging data, a two-dimensional (2D) image slice of a fetus and a three-dimensional (3D) rendering of the fetus, determine an orientation of the 3D rendering based on one or more anatomical features of the fetus, responsive to determining that the orientation is not a standard orientation, adjust the orientation to the standard orientation, and simultaneously display, via the display device, the 2D image slice and the 3D rendering in the standard orientation. In a first example of the system, determining the orientation of the 3D rendering based on the one or more anatomical features of the fetus includes searching for the one or more anatomical features in the 3D rendering, responsive to the one or more anatomical features being identified, determining a vertical axis of the fetus based on the one or more anatomical features, and determining the orientation of the 3D rendering with respect to the vertical axis. In a second example of the system, optionally including the first example, the one or more anatomical features comprise a nose and a mouth, and the vertical axis bifurcates the nose and the mouth. In a third example of the system, optionally including one or more of the first and second examples, the one or more anatomical features comprise a nose or a mouth, and determining the vertical axis based on the one or more anatomical features includes determining a transverse axis based on the one or more anatomical features, and generating a vertical axis perpendicular to the transverse axis and bifurcating the nose or the mouth. In a fourth example of the system, optionally including one or more of the first through third examples, the one or more anatomical features further comprise eyes or ears, and the transverse axis bifurcates the eyes or the ears. In a fifth example of the system, optionally including one or more of the first through fourth examples, determining the orientation is not the standard orientation includes the orientation being outside of a threshold angle of the standard orientation. In a sixth example of the system, optionally including one or more of the first through fifth examples, the threshold angle is 20°.

In yet another embodiment, a method comprises acquiring imaging data of a fetus from a probe of an ultrasound imaging system, generating, from the imaging data, a two-dimensional (2D) image slice depicting the fetus and a three-dimensional (3D) rendering depicting the fetus, automatically identifying one or more anatomical features of the fetus depicted in the 3D rendering, automatically determining an orientation of the 3D rendering based on the one or more identified anatomical features, responsive to the orientation of the 3D rendering being in a standard orientation, maintaining the orientation of the 3D rendering, responsive to the orientation of the 3D rendering not being in the standard orientation, automatically reversing the orientation of the 3D rendering, and thereafter simultaneously displaying, via a display device of the ultrasound imaging system, the 2D image slice and the 3D rendering. In a first example of the method, the one or more anatomical features comprise one or more facial features. In a second example of the method, optionally including the first example, the method further comprises, following simultaneously displaying the 2D image slice and the 3D rendering, and responsive to a user request received at a user interface of the ultrasound imaging system, updating the orientation of the 3D rendering. In a third example of the method, optionally including one or more of the first and second examples, the standard orientation is an upwards orientation relative to the display device.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
acquiring imaging data from a probe of an ultrasound imager;
generating, from the imaging data, an image slice and a rendering;
determining an orientation of the rendering;
responsive to determining the orientation not being a standard orientation, adjusting the orientation to the standard orientation; and
displaying the image slice unaltered while providing the rendering in the standard orientation.

2. The method of claim 1, wherein
the imaging data includes fetal imaging data; and
each of the image slice and the rendering depict one or more anatomical features of a fetus.

3. The method of claim 2, wherein
the one or more anatomical features comprise one or more facial features; and
the standard orientation is an upwards orientation relative to a vertical axis determined from the one or more facial features.

4. The method of claim 2, wherein determining the orientation of the rendering includes:
identifying the one or more anatomical features; and
determining the orientation based on the one or more identified anatomical features.

5. The method of claim 4, wherein identifying the one or more anatomical features includes using a system of deep neural networks to identify the one or more anatomical features from the rendering.

6. The method of claim 5, wherein, prior to identifying the one or more anatomical features from the rendering, the system of deep neural networks is trained with a training set of additional renderings depicting one or more anatomical features of further fetuses.

7. The method of claim 1, further comprising:
responsive to a position of the probe of the ultrasound imager being altered, automatically adjusting the orientation to the standard orientation in real time.

8. The method of claim 2, further comprising:
responsive to a position of the probe of the ultrasound imager being altered:
responsive to the altered position being outside a detection range of the fetus, generating and displaying a notification; and
responsive to the altered position being inside the detection range of the fetus, adjusting the orientation to the standard orientation.

9. The method of claim 1, further comprising:
receiving a user request for an updated orientation;
adjusting the orientation to the updated orientation; and
displaying the rendering in the updated orientation.

10. A system, comprising:
an ultrasound probe;
a user interface configured to receive input from a user of the system;
a display device; and
a processor configured with instructions in non-transitory memory that when executed cause the processor to:
acquire fetal imaging data from the ultrasound probe;
generate, from the fetal imaging data, a two-dimensional (2D) image slice of a fetus and a three-dimensional (3D) rendering of the fetus;
determine an orientation of the 3D rendering based on one or more anatomical features of the fetus;
responsive to determining that the orientation is not a standard orientation, adjust the orientation to the standard orientation; and
simultaneously display, via the display device, the 2D image slice and the 3D rendering in the standard orientation.

11. The system of claim 10, wherein determining the orientation of the 3D rendering based on the one or more anatomical features of the fetus includes:
searching for the one or more anatomical features in the 3D rendering;
responsive to the one or more anatomical features being identified:
determining a vertical axis of the fetus based on the one or more anatomical features; and
determining the orientation of the 3D rendering with respect to the vertical axis.

12. The system of claim 11, wherein
the one or more anatomical features comprise a nose and a mouth; and
the vertical axis bifurcates the nose and the mouth.

13. The system of claim 11, wherein
the one or more anatomical features comprise a nose or a mouth; and
determining the vertical axis based on the one or more anatomical features includes:
determining a transverse axis based on the one or more anatomical features; and
generating a vertical axis perpendicular to the transverse axis and bifurcating the nose or the mouth.

14. The system of claim 13, wherein
the one or more anatomical features further comprise eyes or ears; and
the transverse axis bifurcates the eyes or the ears.

15. The system of claim 11, wherein determining the orientation is not the standard orientation includes the orientation being outside of a threshold angle of the standard orientation.

16. The system of claim 15, wherein the threshold angle is 20°.

17. A method for an ultrasound imaging system, comprising:
acquiring imaging data of a fetus from a probe of an ultrasound imaging system;
generating, from the imaging data, a two-dimensional (2D) image slice depicting the fetus and a three-dimensional (3D) rendering depicting the fetus;
automatically identifying one or more anatomical features of the fetus depicted in the 3D rendering;
automatically determining an orientation of the 3D rendering based on the one or more identified anatomical features;
responsive to the orientation of the 3D rendering being in a standard orientation, maintaining the orientation of the 3D rendering;
responsive to the orientation of the 3D rendering not being in the standard orientation, automatically reversing the orientation of the 3D rendering; and thereafter
simultaneously displaying, via a display device of the ultrasound imaging system, the 2D image slice and the 3D rendering.

18. The method of claim 17, wherein the one or more anatomical features comprise one or more facial features.

19. The method of claim 17, further comprising:
following simultaneously displaying the 2D image slice and the 3D rendering, and responsive to a user request received at a user interface of the ultrasound imaging system, updating the orientation of the 3D rendering.

20. The method of claim 17, wherein the standard orientation is an upwards orientation relative to the display device.
